(19) United States
(12) Patent Application Publication
(10) Pub. No.: US 2020/0055961 A1
(43) Pub. Date: Feb. 20, 2020

(54) PREPARATION OF OXIDIZED CELLULOSE, AND APPLICATION OF OXIDIZED CELLULOSE IN WASHING AND DRUG LOADING

(71) Applicant: CHINA AGRICULTURAL UNIVERSITY, Beijing (CN)

(72) Inventors: Yuan LI, Beijing (CN); Bin LIU, Beijing (CN)

(21) Appl. No.: 16/606,515

(22) PCT Filed: Apr. 21, 2017
(86) PCT No.: PCT/CN2017/081516

§ 371 Date: Oct. 18, 2019

(51) Int. Cl. C08B 15/04; C11D 3/22; A61K 47/38

(52) U.S. Cl. CPC C08B 15/04; A61K 47/38; C11D 3/223

(57) ABSTRACT

A method for preparing oxidized cellulose, oxidized cellulose, an application of oxidized cellulose in manufacturing a detergent, and a detergent. The method comprises: preforming extraction on a corncob to obtain cellulose; preparing the cellulose into a cellulose solution; and performing TEMPO oxidation on the cellulose solution to obtain oxidized cellulose.

### TECHNICAL FIELD

The present disclosure relates to the fields of medicine and chemical industries, and more particularly to the preparation of oxidized cellulose and use of the oxidized cellulose in cleaning and drug loading applications. More specifically, the present disclosure relates to a method for preparing oxidized cellulose, oxidized cellulose, use of oxidized cellulose in preparation of a detergent, a probe or a drug carrier, and a detergent having the oxidized cellulose.

### BACKGROUND

Cellulose is a kind of natural polymer with abundant natural resources, which is renewable and environment-friendly. At present, the cellulose can be modified by artificial means to change its structure or physical and chemical properties, so as to improving its application value.

However, current methods for modifying cellulose still need to be improved.

### SUMMARY

Embodiments of the present disclosure seek to solve at least one of the problems existing in the related art to at least some extent. For this, embodiments of the present disclosure provide a method for preparing oxidized cellulose, oxidized cellulose, use of oxidized cellulose in preparation of a detergent, a probe or a drug carrier, and a detergent having the oxidized cellulose. The oxidized cellulose obtained by the preparation method according to embodiments of the present disclosure has a spherical structure, good amphipathy, strong emulsification, low toxicity, and high application value.

It should be noted that, the present disclosure is based on the following findings of inventors.

At present, TEMPO (2,2,6,6-tetramethylpiperidinyl-N-oxide) oxidation technology of cellulose has been applied to cellulose modification, which mainly uses a TEMPO-NaClO—NaBr system to oxidize a C6 primary hydroxyl group of the cellulose to a carboxyl group. However, the cellulose after oxidized still has a micron-sized chain structure, poor hydrophilicity and no emulsification effect.

In view of this, the inventors have unexpectedly discovered that the source of the cellulose has a great influence on the structure of TEMPO-oxidized cellulose. When corncob-derived cellulose is used as a raw material for TEMPO oxidation, and reaction conditions of the TEMPO oxidation, for example the addition amount of an alkali solution for maintaining the pH needed by the reaction substantially constant, a reaction temperature, etc., are changed at the same time, the resulting oxidized cellulose may have a spherical structure with a particle size ranging from 20 to 30 nm, and become hydrophilic from original hydrophobic cellulose, as well as has a strong emulsification, low toxicity, and high application value.

Therefore, in a first aspect, embodiments of the present disclosure provide a method for preparing oxidized cellulose, including: extracting cellulose from corncob; preparing the cellulose into a cellulose-contained solution; and performing TEMPO oxidation on the cellulose-contained solution, so as to obtain the oxidized cellulose. The TEMPO oxidation on the cellulose-contained solution includes: (1) mixing TEMPO and NaBr with the cellulose-contained solution, and adjusting pH to 10 with a first NaOH solution, so as to obtain a mixture; (2) adding a NaClO solution and a second NaOH solution sequentially and alternately to the mixture obtained in step (1) so as to maintain pH of 10; and (3) adding ethanol to a reaction mixture obtained in step (2) to terminate the reaction, and adding NaBH after 3 to 5 min, so as to obtain the oxidized cellulose. The TEMPO oxidation is performed at a temperature ranging from 15 to 25° C.

The inventors have found that the reaction temperature of the TEMPO oxidation has significant effects on the particle size, structure or yield of the oxidized cellulose. Further, the inventors have found through extensive experiments that when the reaction temperature is in the range of 15 to 25° C., spherical cellulose with a nano-sized particle size can be obtained at a high yield, and the spherical cellulose has both hydrophilic and lipophilic properties. Therefore, the oxidized cellulose obtained by the method for preparing oxidized cellulose according to embodiments of the present disclosure has a spherical structure, small particle size, good amphipathy, and low toxicity.

According to some embodiments of the present disclosure, the oxidized cellulose described above may also have the following additional technical features.

In some embodiments, based on 1 g of the cellulose, 5 to 12.5 mL, preferably 6.25 mL of the second NaOH solution is consumed in the TEMPO oxidation. As a result, the oxidized cellulose obtained by the method for preparing oxidized cellulose according to embodiments of the present disclosure has a spherical structure, small particle size, better hydrophilic and lipophilic properties, and low toxicity.

In some embodiments, based on 1 g of the cellulose, 5 to 16 mL of the NaClO solution is consumed in the TEMPO oxidation. As a result, the oxidized cellulose obtained by the method for preparing oxidized cellulose according to embodiments of the present disclosure has a spherical structure, small particle size, better hydrophilic and lipophilic properties, and low toxicity.

In some embodiments, based on 1 g of the cellulose, the TEMPO is used in an amount ranging from 0.005 to 0.010 g, preferably 0.008 g. As a result, the oxidized cellulose obtained by the method for preparing oxidized cellulose according to embodiments of the present disclosure has a spherical structure, small particle size, better hydrophilic and lipophilic properties, and low toxicity.

In some embodiments, based on 1 g of the cellulose, the NaBr is used in an amount ranging from 0.2 to 0.5 g, preferably 0.4 g. As a result, the oxidized cellulose obtained by the method for preparing oxidized cellulose according to embodiments of the present disclosure has a spherical structure, small particle size, better hydrophilic and lipophilic properties, and low toxicity.

In some embodiments, the method further includes a purification treatment. The purification treatment includes: adjusting an oxidized cellulose-contained reaction mixture obtained in step (3) with a hydrochloric acid solution to pH of 3, and performing a first mixing treatment, so as to obtain a first purified solution; adjusting the first purified solution with a third NaOH solution to pH of 7, and performing a second mixing treatment, so as to obtain a second purified solution; adding ethanol to the second purified solution, followed by collecting, washing with ethanol and acetone in sequence, and drying precipitates, so as to obtain the oxidized cellulose. As a result, the oxidized cellulose obtained by the method for preparing oxidized cellulose according to embodiments of the present disclosure has a spherical structure, small particle size, better hydrophilic and lipophilic properties, and low toxicity.

In some embodiments, extracting cellulose from corncob includes: subjecting the corncob to steam explosion and washing corncob residue obtained thereby; and adding a NaOH solution to the corncob residue to obtain a mixture, followed by heating the mixture, separating solids from the mixture, collecting and washing the solids, so as to obtain the cellulose. As a result, the oxidized cellulose obtained by the method for preparing oxidized cellulose according to embodiments of the present disclosure has a spherical structure, small particle size, better hydrophilic and lipophilic properties, and low toxicity.

In a second aspect, embodiments of the present disclosure provide an oxidized cellulose. The oxidized cellulose is prepared by a method for preparing oxidized cellulose according to any embodiments of the first aspect of the present disclosure described hereinbefore. Therefore, the oxidized cellulose according to some embodiments of the present disclosure has a spherical structure, small particle size, better hydrophilic and lipophilic properties, and low toxicity.

In some embodiments, the oxidized cellulose is spherical with a particle size ranging from 20 to 30 nm. Therefore, the oxidized cellulose according to some embodiments of the present disclosure has a spherical structure, small particle size, better hydrophilic and lipophilic properties, and low toxicity.

In a third aspect, embodiments of the present disclosure provide use of the oxidized cellulose as described above in preparation of a detergent, a probe or a drug carrier. The oxidized cellulose according to some embodiments of the present disclosure has a small particle size, better hydrophilic and lipophilic properties, strong emulsification and low toxicity, and thus can be used in preparation of the detergent, the probe or drugs.

In a fourth aspect, embodiments of the present disclosure provide a detergent including the oxidized cellulose described above. Therefore, the detergent according to embodiments of the present disclosure has better cleaning and decontaminating effects.

In some embodiments, the detergent is used at 1 to 10 mg/l mL water. Thus, the detergent according to embodiments of the present disclosure has further improved cleaning and decontaminating effects.

Additional aspects and advantages of embodiments of present disclosure will be given in part in the following descriptions, become apparent in part from the following descriptions, or be learned from the practice of the embodiments of the present disclosure.

### BRIEF DESCRIPTION OF THE DRAWINGS

These and other aspects and advantages of embodiments of the present disclosure will become apparent and more readily appreciated from the following descriptions made with reference to the drawings, in which:

FIG. 1 shows a flow chart of a method for preparing oxidized cellulose according to an embodiment of the present disclosure;

FIG. 2 shows a transmission electron micrograph of oxidized cellulose according to an embodiment of the present disclosure;

FIG. 3 shows an atomic force micrograph of oxidized cellulose according to an embodiment of the present disclosure;

FIG. 4 shows a scanning electron micrograph of oxidized cellulose according to an embodiment of the present disclosure;

FIG. 5 is a schematic diagram showing effects of different addition amounts of a NaOH solution according to an embodiment of the present disclosure;

FIG. 6 is a schematic diagram showing contact angle analysis according to an embodiment of the present disclosure;

FIG. 7 is a diagram showing effects of different TEMPO addition amounts according to an embodiment of the present disclosure;

FIG. 8 is a diagram showing effects of different NaBr addition amounts according to an embodiment of the present disclosure;

FIG. 9 shows a scanning electron micrograph of oxidized cellulose according to another embodiment of the present disclosure;

FIG. 10 shows a scanning electron micrograph of oxidized cellulose obtained from wood-derived cellulose according to yet another embodiment of the present disclosure;

FIG. 11 is a schematic diagram showing an analysis of dish cleaning experiments according to an embodiment of the present disclosure;

FIG. 12 shows graphs of toxicity experiments according to an embodiment of the present disclosure;

FIG. 13 shows a flow chart of a method for preparing a drug-loaded oxidized cellulose carrier according to an embodiment of the present disclosure;

FIG. 14 is a schematic diagram showing an analysis of cytotoxicity experiments according to an embodiment of the present disclosure;

FIG. 15 is a schematic diagram showing an analysis of cellular uptake experiments according to an embodiment of the present disclosure;

FIG. 16 is a schematic diagram showing plant growth according to an embodiment of the present disclosure;

FIG. 17 is a schematic diagram showing an analysis of phytotoxicity experiments according to an embodiment of the present disclosure; and

FIG. 18 is a schematic diagram showing a reaction process according to an embodiment of the present disclosure.

### DETAILED DESCRIPTION

In the following, embodiments of the present disclosure will be described in detail. It will be appreciated that embodiments described hereinafter are explanatory, illustrative, and used to generally understand the present disclosure, and shall not be construed to limit the present disclosure.

It should be noted that, terms such as “first” and “second” are used herein merely for purposes of description and are not intended to indicate or imply relative importance or significance or to imply the number of indicated technical features. Thus, the feature defined with “first” and “second” may include one or more of this feature. Further, in the description of the present disclosure, a phrase of “a plurality of” means two or more than two, unless specified otherwise.

Embodiments of the present disclosure provide a method for preparing oxidized cellulose, oxidized cellulose, use of oxidized cellulose in preparation of a detergent, a probe or a drug carrier, and a detergent having the oxidized cellulose, which will be illustrated in detail below, respectively.

Method for Preparing Oxidized Cellulose

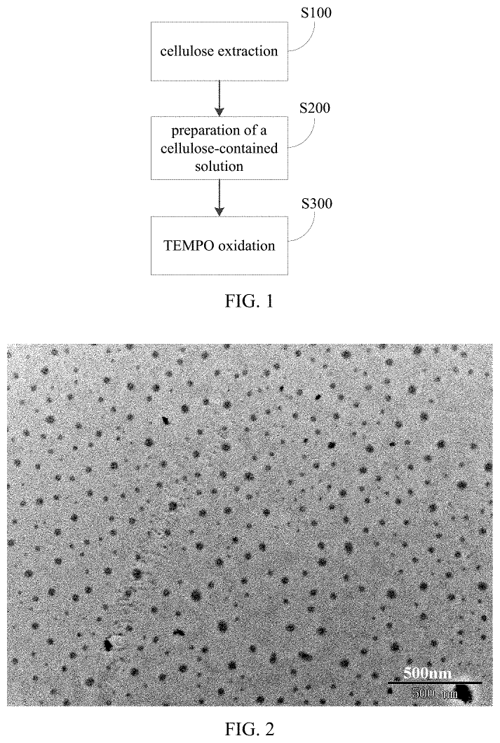

In a first aspect, embodiments of the present disclosure provide a method for preparing oxidized cellulose. As illustrated in FIG. 1, the method includes: cellulose extraction S100, preparation of a cellulose-contained solution S200, and TEMPO oxidation S300. The oxidized cellulose obtained by the method for preparing oxidized cellulose according to some embodiments of the present disclosure has a spherical structure, small particle size, good amphipathy, strong emulsification, and low toxicity. In the following, the method will be descried in detail.

In some embodiments of the present disclosure, the method includes the following operations.

Cellulose Extraction S100

In this step, the cellulose is extracted from corncob. The extracted cellulose is linear and hydrophobic.

The inventors have unexpectedly discovered that nano-sized oxidized cellulose spheroidal particles can be obtained by the TEMPO oxidation of the corncob-derived cellulose, and the obtained oxidized cellulose has good amphipathy; while results obtained from celluloses derived from other sources, such as straws, cottons, are poor. For example, the inventors have found that even subjected to the TEMPO oxidation, the cellulose extracted from the straws is still in a linear structure and almost has no hydrophilicity.

In some embodiments of the present disclosure, the cellulose extraction includes: subjecting the corncob to steam explosion and washing corncob residue obtained thereby; adding a NaOH solution to the corncob residue to obtain a mixture, followed by heating the mixture, separating solids from the mixture, collecting and washing the solids, so as to obtain the cellulose. In this way, the yield of the cellulose is high.

Preparation of the Cellulose-Contained Solution S200

In this step, the cellulose is prepared into the cellulose-contained solution.

In some embodiments of the present disclosure, the cellulose is dissolved in boiling water to obtain a cellulose aqueous solution.

TEMPO Oxidation S300

In this step, the cellulose-contained solution is subjected to the TEMPO oxidation to obtain the oxidized cellulose.

It should be noted that, embodiments of the present disclosure employ a TEMPO-NaClO—NaBr system for the TEMPO oxidation. The main principle lies in that, the NaClO, as a main oxidant of the process, first reacts with the NaBr to form NaBrO, and then the NaBrO oxidizes the TEMPO to a nitrosonium ion. The nitrosonium ion oxidizes a primary alcohol hydroxyl group into an aldehyde group (intermediate), which is eventually oxidized into a carboxyl group (FIG. 18).

In some embodiments of the present disclosure, the TEMPO oxidation on the cellulose-contained solution includes the following operations.

(1) The TEMPO and NaBr are mixed with the cellulose-contained solution, and a solution obtained thereby is adjusted by a first NaOH solution to pH of 10, so as to obtain a mixture.

The inventors have found that by adjusting the pH of the solution with the NaOH solution, a reaction condition required for the TEMPO oxidation, i.e., the pH of 10, may be achieved.

(2) A NaClO solution and a second NaOH solution are added sequentially and alternately to the mixture obtained in step (1) so as to maintain pH of 10.

The inventors have found that an oxidized degree of the cellulose will significantly affect the structure, hydrophilicity and yield of the resulting product. Further, the inventors have found that if the NaClO solution is totally added to the mixture obtained in step (1) at one time, the yield of the oxidized cellulose is low, and even the oxidation of the cellulose cannot be achieved. Therefore, in embodiments of the present disclosure, the NaClO solution is gradually added in batches so as to control the oxidized degree of the cellulose. With the oxidation reaction proceeds, the pH of the reaction system will decrease. When the pH of the reaction system begins to decrease, the addition of the NaClO solution is stopped, replaced by addition of the second NaOH solution until the pH of the reaction solution returns to 10. Then, the second NaOH solution is stopped to add, and the NaClO solution is added again. Like this, the NaClO solution and the second NaOH solution are alternately and repeatedly added, so as to basically maintain the pH of the reaction system at 10.

In some embodiments of the present disclosure, based on 1 g of the cellulose, the second NaOH solution consumed in the TEMPO oxidation is in a range of 5 to 12.5 mL, preferably 6.25 mL. The inventors have found that the amount of the second NaOH solution will affect a degree of oxidation, and the degree of oxidation will affect the structure, hydrophilicity and yield of the product. Further, the inventors have found that when the second NaOH solution used in the TEMPO oxidation is 5 to 12.5 mL, the degree of oxidation is 40% to 100%, and the oxidation effect is better. Under this condition, nano-sized spherical cellulose particles can be obtained with high yield, and the obtained spherical cellulose particles have hydrophilic and lipophilic properties and good emulsification. Specifically, when the second NaOH solution used in the TEMPO oxidation is 5 mL, the degree of oxidation is 40% (DO40); when the second NaOH solution used in the TEMPO oxidation is 6.25 mL, the degree of oxidation is 50% (DO50); when the second NaOH solution used in the TEMPO oxidation is 12.5 mL, the degree of oxidation is 100% (DO100). When the second NaOH solution used in the TEMPO oxidation is 6.25 mL, the resulting oxidized cellulose has a better amphipathy and strong emulsification. Therefore, the oxidized cellulose obtained by the method for preparing oxidized cellulose according to embodiments of the present disclosure has a spherical structure, small particle size, better hydrophilic and lipophilic properties, and low toxicity.

In addition, it should be illustrated that, the amount of each of the TEMPO, NaBr, and NaClO also affects the structure, hydrophilicity and yield of the product, especially the particle size of the formed spherical cellulose particles. In some embodiments of the present disclosure, based on 1 g of the cellulose, 5 to 16 mL of the NaClO solution, 0.005 to 0.010 g, preferably 0.008 g of the TEMPO, and 0.2 to 0.5 g, preferably 0.4 g of the NaBr are consumed in the TEMPO oxidation. The above preferred dosage ratio is obtained by the inventors through a large number of experiments. Under these conditions, nano-sized spherical cellulose particles can be obtained with high yield, and the obtained spherical cellulose particles have hydrophilic and lipophilic properties and good emulsification. Therefore, the oxidized cellulose obtained by the method for preparing oxidized cellulose according to embodiments of the present disclosure has a spherical structure, small particle size, better hydrophilic and lipophilic properties, and low toxicity.

(3) Ethanol is added to a reaction mixture obtained in step (2) to terminate the reaction, and after 3 to 5 min, NaBH is added thereto, so as to obtain the oxidized cellulose.

In some embodiments of the present disclosure, the method further includes a purification treatment, which includes the following operations.

(i) An oxidized cellulose-contained reaction mixture obtained in step (3) is adjusted with a hydrochloric acid solution to pH of 3, and a first mixing treatment is performed, so as to obtain a first purified solution. The inventors have unexpectedly found that if the reaction mixture obtained in step (3) is directly subjected to alcohol precipitation, the residual NaBr will affect the yield of the oxidized cellulose, resulting in a decrease in the yield. However, the inventors have found that by adjusting the reaction mixture with the acid solution to the pH of 3, the NaBr can be effectively neutralized, thereby improving the yield.

(ii) The first purified solution is adjusted with a third NaOH solution to pH of 7, and a second mixing treatment is performed, so as to obtain a second purified solution. In this way, the hydrochloric acid solution added in the previous step is neutralized by the NaOH solution.

(iii) Ethanol is added to the second purified solution, followed by collecting, washing with ethanol and acetone in sequence, and drying precipitates, so as to obtain the oxidized cellulose. The inventors have found that, if the precipitates are dried directly after washed by ethanol, while without further washed by acetone, agglomeration is easy to occur. The oxidized cellulose once agglomerated will be difficult to observe, thereby easily resulting in misjudgment to the particle size thereof. However, using the acetone to further wash the precipitate, the ethanol can be washed away.

In some embodiments of the present disclosure, the TEMPO oxidation is performed at a temperature ranging from 15 to 25° C. That is, the steps (1) to (3) each are performed at the temperature of 15 to 25° C. The inventors have found that the reaction temperature of the TEMPO oxidation will significantly affect the particle size, structure, yield and activity of the oxidized cellulose. Further, the inventors have found through extensive experiments that when the reaction temperature is in the range of 15 to 25° C., spherical cellulose with nano-sized particle size can be obtained at a high yield, and the spherical cellulose has hydrophilic and lipophilic properties and strong emulsification, and thus may be used as a surfactant. However, results obtained under other reaction temperatures are not good, for example, it is unable to obtain spherical cellulose or the particle size thereof is too large to reach a nanoscale; the oxidation is unsuccessful, so that the spherical oxidized cellulose cannot be obtained; or the yield is low. Therefore, the oxidized cellulose obtained by the method for preparing oxidized cellulose according to embodiments of the present disclosure has a spherical structure, small particle size, good amphipathy, strong emulsification and low toxicity.

It should be illustrated that the term “spherical” as used herein should be broadly understood, and may include regular structures, such as spherical, ellipsoidal, hemispherical structures, and irregular spherical structures.

In addition, it should be illustrated that the term “nano-sized” as used herein refers to particles having a particle size between 1 nm and 100 nm.

Furthermore, it should be noted that the terms “linear” and “spherical” as used herein are related to microstructures of substances, such as that observed under a scanning electron microscope.

Oxidized Cellulose

In a second aspect, embodiments of the present disclosure provide an oxidized cellulose.

In some embodiments, the oxidized cellulose is prepared by the method for preparing oxidized cellulose according to any embodiment in the first aspect of the present disclosure as described hereinbefore. Therefore, the oxidized cellulose according to embodiments of the present disclosure has a spherical structure, small particle size, better hydrophilic and lipophilic properties, or low toxicity.

In some embodiments of the present disclosure, the oxidized cellulose is spherical with a particle size ranging from 20 to 30 nm. Therefore, the oxidized cellulose according to embodiments of the present disclosure has a spherical structure, small particle size, better hydrophilic and lipophilic properties, or low toxicity.

It will be appreciated to those skilled in the art that, characteristics and advantages described above with respect to the method for preparing oxidized cellulose are also applicable to the oxidized cellulose described here, and thus will not be elaborated again.

Use

In a third aspect, embodiments of the present disclosure provide use of the oxidized cellulose as described hereinbefore in preparation of a detergent, a probe or a drug carrier.

Because of the better hydrophilic and lipophilic properties and strong emulsification, the oxidized cellulose according to embodiments of the present disclosure may be used as a surfactant to adsorb oils and stains on cloth, and a decontamination effect thereof is better. Moreover, due to low toxicity, the oxidized cellulose according to embodiments of the present disclosure has no irritation to skin. In addition, owing to the better hydrophilic and lipophilic properties and the smaller particle size (nano-sized), the oxidized cellulose according to embodiments of the present disclosure is able to penetrate a cell membrane, so that it may be used as a carrier to prepare the probe or the drug carrier for better detection or therapeutic purposes, and the safety is high.

It will be appreciated to those skilled in the art that, characteristics and advantages described above with respect to the oxidized cellulose are also applicable to the use thereof as described here, and thus will not be elaborated again.

Detergent

In a fourth aspect, embodiments of the present disclosure provide a detergent.

In some embodiments of the present disclosure, the detergent includes the oxidized cellulose described hereinbefore. Therefore, the detergent according to embodiments of the present disclosure has better cleaning and decontaminating effects.

In some embodiments of the present disclosure, the detergent is used at 1 to 10 mg/l mL water. According to a specific embodiment, when used for washing, 1 to 10 mg of the detergent may be dissolved in per milliliter of water. In this way, the detergent according to embodiments of the present disclosure further has the better cleaning and decontaminating effects.

It will be appreciated to those skilled in the art that, characteristics and advantages described above with respect to the oxidized cellulose are also applicable to the detergent as described here, and thus will not be elaborated again.

The present disclosure will be further explained with reference to the following examples. It will be appreciated to those skilled in the art that the following examples are illustrative, and merely used to generally understand the present disclosure, and shall not be construed to limit the present disclosure. Examples where specific techniques or conditions are not indicated are carried out according to those described in literatures of the related art or in accordance with product specifications. Reagents or instruments without indicating manufacturers are conventional products which are commercially available.

### Example 1

In this example, the oxidized cellulose is prepared in accordance with the following method.

1. Steam Explosion of Corncob

100 g of the corncob was weighed with a balance and placed in a 2 L beaker, to which 1000 mL deionized water was added to completely submerge the corncob. After 8 h, water was drained off, and the corncob was subjected to the steam explosion with sulfuric acid steam under an explosion pressure of 0.9 MPa for 5 min. The resulting corncob residue was extracted twice with hot water of 80° C. for 1 h each time, and then washed to neutral and freeze-dried for ready use.

2. Extraction of Cellulose

10 mg of the corncob residue obtained above was weighed and added with 7.5 mL 2% NaOH solution, and then heated at 160° C. for 2 h. After the reaction was finished, the resulting mixture was cooled, and then subjected to solid-liquid separation. The separated solids were washed with deionized water to neutral to obtain the cellulose.

3. TEMPO Oxidation

(1) 1 g of the cellulose was weighed and dissolved in 100 mL boiling water, followed by cooling to a temperature of 8 to 10° C. with ice cube to obtain a cellulose-contained solution.

(2) 0.008 g TEMPO (2,2,6,6-tetramethylpiperidinyl-N-oxide) and 0.4 g NaBr were weighed respectively, and dissolved with a small amount of water (about 1 mL). A solution obtained thereby was added to the cellulose-contained solution, followed by addition of 2 mol/L NaOH solution to adjust pH to 10, and the temperature was kept at 25° C.

(3) To a reaction mixture resulted at step (2) was added a NaClO solution with pH of 10 (calibrated by concentrated hydrochloric acid) to initiate the oxidation reaction. When the pH of the reaction mixture began to decrease, 0.5 mol/L NaOH solution was added dropwise to the reaction mixture to maintain the pH at 10. Like this, the NaClO solution and the NaOH solution were added alternately. The temperature was kept at 25° C.

(4) When the addition amount of the NaOH solution added in step (3) reached 6.25 mL, 2 mL ethanol was added to terminate the reaction. 5 min later, 0.05 g NaBH was added, followed by stirring for 1 h. The temperature was kept at 25° C.

(5) To a reaction mixture resulted at step (4) was added 4 mol/L hydrochloric acid to adjust pH to 3, followed by stirring for 1 h.

(6) To a reaction mixture resulted at step (5) was added 1 mol/L NaOH solution to adjust pH to 7, followed by stirring for 1 h.

(7) Ethanol was added under stirring to a reaction mixture resulted at step (6), with a volume of the ethanol being 1 to 1.5 times that of the reaction mixture. As a result, floccules were produced. The reaction mixture was allowed to stand for 1 h, and then subjected to suction filtration. A resulting filter cake was washed with ethanol for three times and acetone for one time, and then put in a fume cupboard to completely volatilize the acetone to afford the oxidized cellulose.

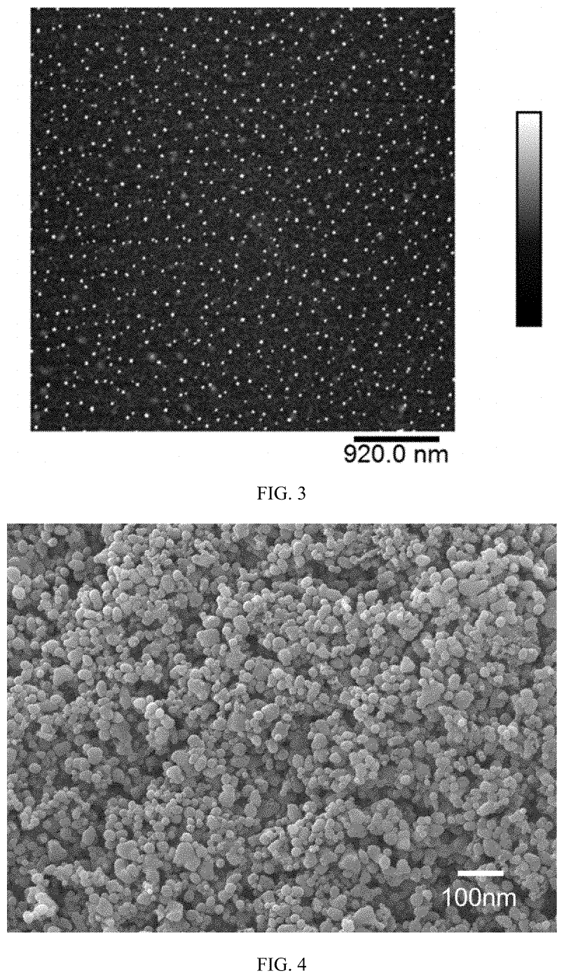

Morphologies of the obtained oxidized cellulose under a transmission electron microscope, an atomic force microscope and a scanning electron microscope are shown in FIG. 2 to FIG. 4, respectively. As can be seen, the obtained oxidized cellulose is spherical with a particle size in a range of 20 to 30 nm.

### Example 2

In this example, effects of the amounts of the NaOH solution added at step (4) were studied.

The specific operations were as follows.

(1) Oxidized celluloses were prepared in three parallel experiments according to the method as described in Example 1, except that the amounts of the NaOH solution added at step (4) were 3.75 mL, 6.25 mL and 12.5 mL, respectively, thereby affording the oxidized celluloses 1 to 3.

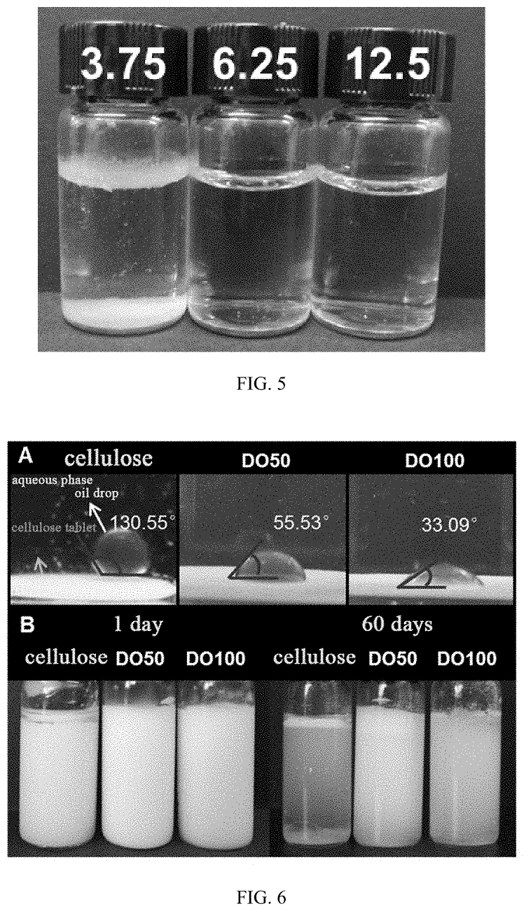

(2) For each of the oxidized celluloses 1 to 3, 2 mg sample was dissolved in 2 mL water to observe its solubility. Results are shown in FIG. 5.

As can be seen, the solubility of the oxidized cellulose 1 prepared with addition of 3.75 mL NaOH solution at step (4) is poor, and with the increase of the NaOH, the solubility of the oxidized cellulose gradually increases.

(3) Contact angles of the oxidized celluloses 2 and 3 were measured as follows.

The contact angle was measured with a pressing disk. An equal amount (40 mg) of the cellulose obtained at step 2 of Example 1, the oxidized cellulose 2 (DO50) and the oxidized cellulose 3 (DO100) were weighed, and after fully dried, were pressed into tablets of the same weight separately by an infrared tablet press with a pressure of 50 kN and a time of 3 min. For each tablet, it was immobilized at a bottom of a beaker containing a certain volume of sunflower seed oil, and then injected thereon with 2 μL deionized water by a gas phase needle. A height of a stage and a focal length of a CCD camera were adjusted until the water droplet can be clearly seen at a computer end. After stabilization, the computer end took photos at a certain rate, and the contact angle at an oil-water interface was determined by software (five-point fitting method). Results are shown in FIG. 6.

Determination of a Degree of Oxidation:

The degree of oxidation was determined by an automatic potentiometric titrator. 50 mg of an analyte to be tested was weighed and dissolved in 50 mL deionized water, followed by addition of a few drops of 1 mol/L NaNO₃ solution to adjust pH to 3. A resulting mixture was titrated with 1 mol/L NaOH solution, with a titration rate of 1 drop per second. The titration was ended when the pH is 7, and a volume of the NaOH solution consumed in the process was recorded. The degree of oxidation of the analyte was calculated with starch having a known degree of oxidation as a reference.

Analysis of Results:

The contact angle characterizes the hydrophilicity of materials. A material has a contact angle less than 90°, indicating that the material is hydrophilic, and the smaller the contact angle is, the stronger the hydrophilicity is. A material has a contact angle greater than 90°, indicating that the material is hydrophobic, and the larger the contact angle is, the stronger the hydrophobicity is. A material has a contact angle approaching 90°, indicating that the material is amphipathic. As can be seen from FIG. 6, a contact angle of the cellulose which is not oxidized is 130.55°, exhibiting a hydrophobicity (lipophilicity); while contact angles of the cellulose after oxidized are less than 90° (55.53° for DO50, and 33.09° for DO100), both exhibiting a hydrophilicity, and with the degree of oxidation increases, the hydrophilicity increases. This is a result of the introduction of carboxyl groups in the TEMPO oxidation process, as the carboxyl group can be dissociated in water, making the original hydrophobic cellulose become hydrophilic, and the hydrophilicity increases as the amount of the carboxyl groups increases. In order to make the obtained oxidized cellulose both hydrophilic and lipophilic, the DO50 was selected as an optimal degree of oxidation, corresponding to the addition amount of the NaOH solution of 6.25 mL.

### Example 3

In this example, effects of the addition amounts of the TEMPO were studied, with the specific operations as follows.

(1) Nanocelluloses were prepared according to the method as described in Example 1, except that the addition amounts of the TEMPO were in a range from 0.05 to 0.10 g, affording different oxidized celluloses.

(2) The yield of the oxidized celluloses was calculated based on the following formula:

Yield of oxidized cellulose (%)=100%×mass of oxidized cellulose obtained at step (7)/1 g.

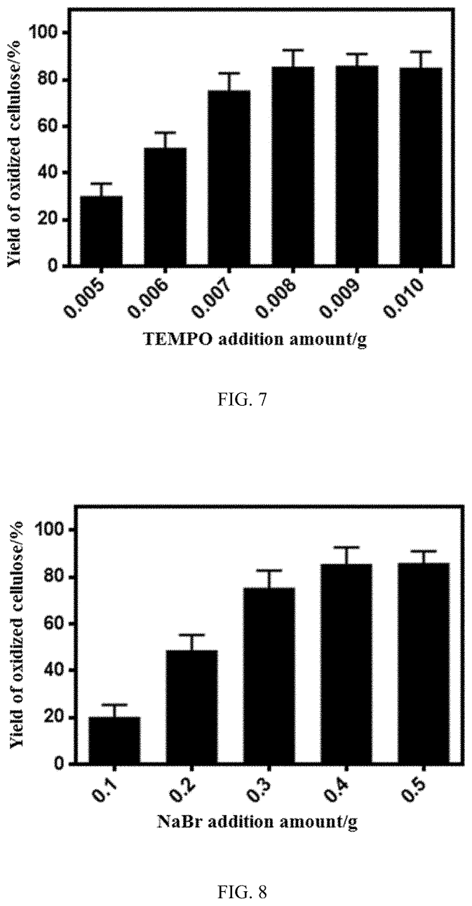

Results are shown in FIG. 7. As can be seen, the best effect is achieved when the addition amount of the TEMPO is 0.008 g.

### Example 4

In this example, effects of the addition amounts of the NaBr were studied, with the specific operations as follows.

(1) Oxidized celluloses were prepared according to the method as described in Example 1, except that the addition amounts of the NaBr were in a range from 0.2 to 0.5 g, affording different oxidized celluloses.

(2) The yield of the oxidized celluloses was calculated based on the following formula:

Yield of oxidized cellulose (%)=100%×mass of oxidized cellulose obtained at step (7)/1 g.

Results are shown in FIG. 8. As can be seen, the best effect is achieved when the addition amount of the NaBr is 0.4 g.

### Example 5

Oxidized cellulose was prepared according to the method as described in Example 1, except that the reaction temperature at steps (2) to (4) was kept at 10° C.

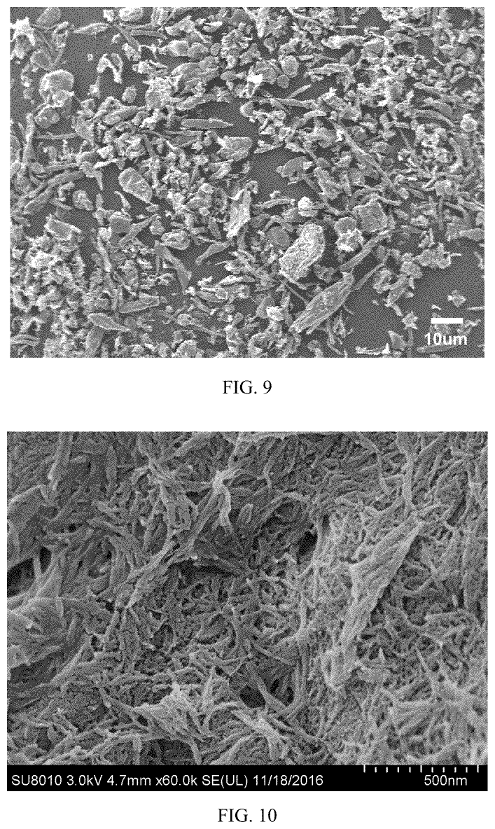

A scanning electron micrograph of the obtained oxidized cellulose is shown in FIG. 9. As can be seen, spherical oxidized cellulose cannot be produced when the reaction temperature is too low.

### Example 6

Oxidized cellulose was prepared according to the method as described in Example 1, except that the corncob is replaced with wood.

A scanning electron micrograph of the obtained oxidized cellulose is shown in FIG. 10. As can be seen, spherical oxidized cellulose cannot be produced when the corncob is replaced with the wood.

### Example 7

The oxidized cellulose prepared in Example 1 was dissolved in water at a concentration of 1 mg/mL to obtain a cleaning solution, and its cleaning effect was verified in the following two manners.

Manner 1: Cloth Cleaning Experiments

Experimental group: a 5×5 cm stained cloth was taken and put into a beaker, to which a certain volume of the above oxidized cellulose-contained solution was added, followed by ultrasonically washing for 10 to 30 min, rinsing twice with clear water, drying and weighing. A weight of the cloth after washed was recorded as m3.

The stained cloth was obtained by dropping a drop of oil, tomato sauce or ink onto a 5×5 cm clean cloth. The clean cloth and the stained cloth were weighted and their weights were recorded as m1 and m2, respectively. Three kinds of cloth were tested, and they are terylene (polyester), silk, and cotton.

Blank group: a 5×5 cm cloth stained with edible oil was taken and put into a beaker, to which a certain volume of water was added, followed by ultrasonically washing for 10 to 30 min, rinsing twice with clear water, drying, and weighing as m3.

Control group: a 5×5 cm cloth stained with edible oil was taken and put into a beaker, to which a certain volume of laundry powder (LP) with a concentration of 1 to 10 mg/mL was added, followed by ultrasonically washing for 10 to 30 min, rinsing twice with clear water, drying, and weighing as m3.

Cleaning rate=(m2−m1)/(m2−m3)

Results are shown in Table 1. It can be seen that, the oxidized cellulose obtained in Example 1 achieves a good decontamination effect, and this is because the oxidized cellulose is able to adsorb and facilitate oils and stains to dissolve in water due to its amphipathy.

TABLE 1
cloth cleaning rate %

| | Edible oil | | | Tomato sauce | | | Black ink | | |
|---|---|---|---|---|---|---|---|---|---|
| Cleaning | | | Laundry | | | Laundry | | | Laundry |
| rate % | Blank | Example 1 | powder | Blank | Example 1 | powder | Blank | Example 1 | powder |
| Terylene | 3.0 | 92.1 | 47.7 | 46.2 | 99.8 | 87.0 | 33.3 | 82.9 | 71.4 |
| Silk | 6.8 | 83.4 | 52.6 | 40.6 | 97.6 | 72.9 | 47.0 | 89.5 | 57.1 |
| Cotton | 10.3 | 91.7 | 64.7 | 48.7 | 98.6 | 80.5 | 41.1 | 86.1 | 43.2 |

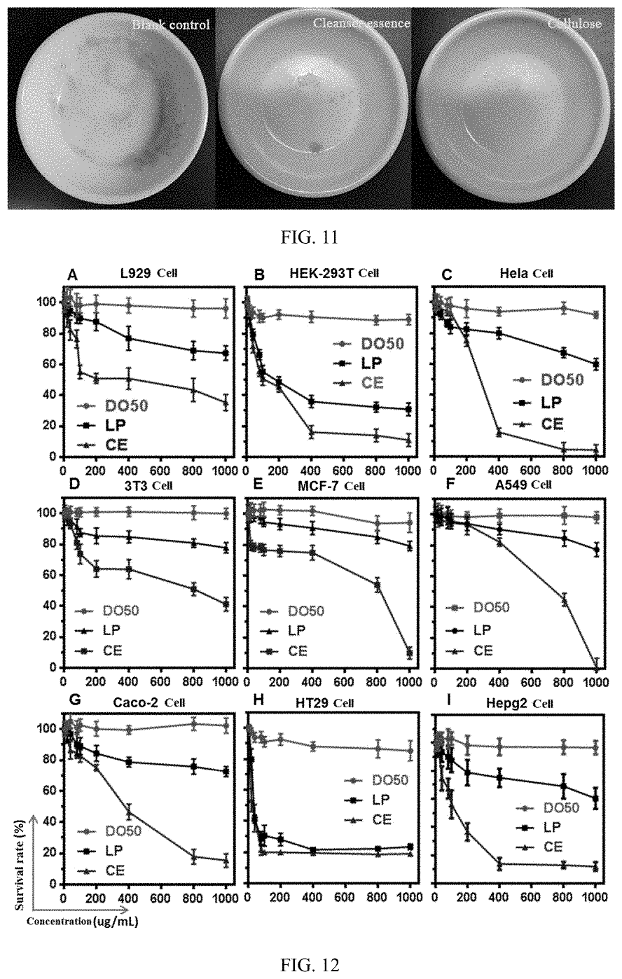

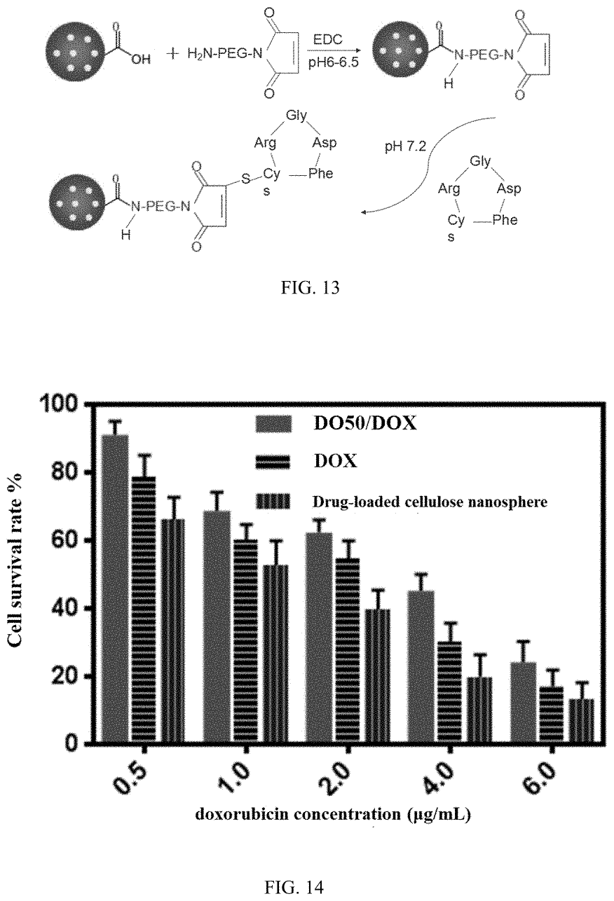

Manner 2: dish cleaning experiments Oil contaminants on dishes were washed with a cleanser essence (CE) and the above cleaning solution separately, with the dish before washed as a blank control. Results are shown in FIG. 11. As can be seen, the oxidized cellulose obtained in Example 1 has a good decontamination effect.

### Example 8

In this example, stabilities of the oxidized celluloses 2 and 3 obtained in Example 2 were tested as follows.

For each of the cellulose obtained at step 2 of Example 1, the oxidized cellulose 2 (DO50) and the oxidized cellulose 3 (DO100) obtained in Example 2, 3 mg of the sample was accurately weighted and dissolved in 3 mL deionized water, followed by further addition of 300 μL sunflower seed oil and vortex mixing evenly. A resulting mixture was prepared into an emulsion by an ultrasonic cell disrupter and placed in a glass bottle to observe its stability.

Analysis of results: the emulsions newly prepared were milky white. After a period of time, the emulsion of the cellulose appeared demulsification, the emulsion of DO100 partially demulsified, and the emulsion of the DO50 did not appear obvious demulsification, indicating that the cellulose cannot stabilize a Pickering emulsion; although DO100 can stabilize the emulsion, its solubility in water is increased due to its high degree of oxidation, which is not conducive to stabilize the Pickering emulsion. However, DO50 has a good emulsification effect.

### Example 9

In this example, the oxidized cellulose obtained in Example 1 was tested for cytotoxicity as follows.

Cells in a logarithmic growth phase were prepared into a single cell suspension, and inoculated in a 96-well culture plate with a volume of 180 μL and a concentration of 1×10⁴ per well. After 24 h of culture at 5% CO₂ and 37° C., 20 μL of the oxidized cellulose (DO50), the laundry powder (LP) or the cleanser essence (CE) was added at a concentration ranging from 20.0 to 1000.0 μg/mL for further culturing 24 h. Subsequently, each well was added with 20 μL CCK-8 reagent. After incubation for another 1 h, an absorbance (A) at a wavelength of 450 nm was determined by an enzyme-linked immune detector. Cell activity is expressed as a ratio (percentage) of the absorbance of the experimental sample to that of the blank control cells.

Results are shown in FIG. 12, as can be seen, the oxidized cellulose (DO50) obtained in Example 1 is non-toxic for both human and murine-derived normal cells (mouse fibroblasts—L929 cells, human embryonic kidney T cells—HEK-293T cells, and mouse embryonic fibroblasts—3T3 cells) and cancer cells (human cervical carcinoma cells—Hela cells, human breast cancer cells—MCF-7, human lung cancer cells—A549, human colon adenocarcinoma cells—CaCo-2, human colon cancer cell—HT29, and human hepatocellular carcinoma cell—Hepg2), while the laundry powder (LP) and the cleanser essence (CE) each have a certain toxicity.

### Example 10

In this example, the oxidized cellulose obtained in example 1 was tested for toxicity on zebrafish.

1. Experimental Materials:

Zebrafish, having a body length in a range of 3.21±0.27 cm, were domesticated for more than 7 days under laboratory conditions, with each day being illuminated for 12 to 16 h. Feces and food residues were removed timely, and a mortality rate was kept below 5%. During the period of domestication, the zebrafish was fed twice a day with readily available bait from the market. The zebrafish was stopped to be fed 24 h before the test and not fed during the test. Healthy and active individuals with uniform body length were selected for the test.

2. Experimental Method: Static Test Method

The oxidized cellulose (DO50), the laundry powder and the cleanser essence were diluted with distilled water to obtain corresponding mother liquors, respectively. For each of the three samples, an appropriate amount of the mother liquor was taken with a pipetting gun and added into a fish tank containing 1 L water, and the volume of liquid in the fish tank was supplemented to 2 L. The solution was stirred to be homogeneous. According to pre-test results, final test concentrations of the samples were set as follows:

DO50: 6, 7, 8, 9, 10 g/L

Laundry powder: 117, 134, 151, 168, 185, 200, 210 mg/L;

Cleanser essence: 40, 50, 60, 70, 80 mg/L.

Test without addition of any sample was taken as a blank control. For each treatment, ten zebrafish was used and three replicates were set up. After 48 and 96 h of the treatment, the temperature, dissolved oxygen and pH value of the solution in the fish tank, as well as the toxic symptoms and mortality of the zebrafish were observed and recorded. The zebrafish, once died, was removed immediately, and its body surface and visceral surface characteristics were observed and recorded.

Water for test was tap water stored and dechlorinated for more than 24 h with pH of 7.5±0.5, a dissolved oxygen content of 8.0±0.5 mg·L⁻¹, a water hardness of 2.4×10² mg·L⁻¹ (measured by CaCO₃) and a temperature of 24±1° C.

3. Data Processing

Experimental results were processed with SPSS 16.0 statistical software. Median lethal concentrations (LC50) and 95% confidence limits of different samples on zebrafish at 48 and 96 h were calculated, a “dose-effect” linear equation was established, and correlation coefficients (R²) were recorded.

Acute toxicities of the samples on the zebrafish were graded based on the standard proposed in “Test guidelines on environmental safety assessment for chemical pesticides”. Reference can be made on OECD. Earthworm, acute toxicity tests. OECD Guideline for testing of chemicals 207[S]. 1984.

Results are shown in tables 2 and 3. It can be seen that the median lethal concentrations (LC50) of the oxidized cellulose on the zebrafish at 2 and 4 days are much higher than that of the laundry powder and the cleanser essence. According to the standard classification, the oxidized cellulose is micro-toxic, the laundry powder is low-toxic, and the cleanser essence is high-toxic.

TABLE 2
Dose-effect relation of DO50, the laundry powder and the
cleanser essence on zebrafish at 2 and 4 days

| | | Correlation | Associated | Statistical |
|---|---|---|---|---|
| Sample | Time/Day | coefficient R² | probability p | probability P |
| DO50 | 2 | 0.985 | 0.002 | 0.989 |
| | 4 | 0.995 | 0.000 | 1.000 |
| Laundry | 2 | 0.943 | 0.001 | 0.932 |
| powder | 4 | 0.983 | 0.000 | 0.960 |
| Cleanser | 2 | 0.964 | 0.008 | 0.925 |
| essence | 4 | 0.933 | 0.021* | 0.972 |

Note:
P > 0.05, accord with normal distribution;
*significant correlation at 0.05 level; others being significantly correlated at 0.01 level.

TABLE 3
Acute toxicity tests of DO50, the laundry powder and
the cleanser essence on zebrafish at 2 and 4 days

| | | Regression | LC₅₀ (95% confidence | Correlation | Toxic |
|---|---|---|---|---|---|
| Sample | Time/day | equation | interval)/(mg · L⁻¹) | coefficient R² | grade |
| DO50 | 2 | Y = −36.318 + 9.184X | 10532 (8158.8~11092.2) | 0.985 | micro- |
| | | | | | toxic |
| | 4 | Y = −35.088 + 8.955X | 8281.5 (7434.2~9527.1) | 0.995 | micro- |
| | | | | | toxic |
| Laundry | 2 | Y = −21.923 + 9.813X | 188.0 (167.9~198.2) | 0.943 | low- |
| powder | | | | | toxic |
| | 4 | Y = −22.522 + 10.214X | 160.4 (146.9~173.7) | 0.983 | low- |
| | | | | | toxic |
| Cleanser | 2 | Y = −20.420 + 11.806X | 63.0 (58.2~68.9) | 0.964 | high- |
| essence | | | | | toxic |
| | 4 | Y = −16.522 + 9.971X | 45.4 (37.2~50.6) | 0.933 | high- |
| | | | | | toxic |

Note:
Y is the mortality rate;
X is a logarithm of the sample concentration;
R² is the correlation coefficient of the regression equation.

### Example 11

In this example, drug loading of the oxidized cellulose obtained in Example 1 was studied as follows.

1. Preparation of Oxidized Cellulose/Doxorubicin (DO50/DOX)

10 mg nanospheres (DO50) obtained in Example 1 was dissolved in water, and then 1 mg doxorubicin hydrochloride (excess) was added thereto. The reaction was fully performed at room temperature for 1 h. Unadsorbed doxorubicin hydrochloride was removed by centrifugation using a 30 KD ultrafiltration tube, and the ultrafiltration tube was washed with water for 3 times.

2. Preparation of Drug-Loaded Oxidized Cellulose

(1) Polyethylene glycol (PEG) modification

10 mg DO50/DOX was dissolved in 5 mL water, and the pH was adjusted to 6, followed by addition of 7 mg 1-(3-dimethylaminopropyl)-3-ethylcarbodiimide hydrochloride (EDC) and 6 mg PEG (PEG is 10% of a molar amount of carboxyl groups of the DO50, and is modified by maleimide in advance) in succession. The reaction was performed at room temperature for 6 h. Then, the EDC and unreacted PEG were removed by centrifugation using a 30 KD ultrafiltration tube, and the ultrafiltration tube was washed with water for 3 times (8000 rpm, 12 min), affording nanospheres coupled with a PEG chain.

(2) Arginyl-Glycyl-Aspartic Acid (RGD) Modification

The nanospheres coupled with the PEG chain obtained above were dissolved in ultrapure water, and then 1.2 mg cyclic RGD was added thereto. The reaction was carried out at room temperature for 2 h. Then, the unreacted RGD was removed by centrifugation using a 30 KD ultrafiltration tube, and the ultrafiltration tube was washed with water for 3 times (8000 rpm, 12 min) to obtain the drug-loaded oxidized cellulose coupled with the cyclic RGD.

3. HepG2 Cytotoxicity Test

Cells in a logarithmic growth phase were prepared into a single cell suspension, and inoculated in a 96-well culture plate with a volume of 180 μL and a concentration of 1×10⁴ per well. After 24 h of culture at 5% CO₂ and 37° C., 20 μL of the oxidized cellulose/doxorubicin (DO50/DOX), the doxorubicin (DOX) or the drug-loaded oxidized cellulose was added at a concentration ranging from 0.5 to 6.0 μg/mL for further culturing 24 h. Subsequently, each well was added with 20 μL CCK-8 reagent. After incubation for another 1 h, an absorbance (A) at a wavelength of 450 nm was determined by an enzyme-linked immune detector. Cell activity is expressed as a ratio (percentage) of the absorbance of the experimental sample to that of the blank control cells.

The cytotoxicity of different nano-carriers was detected by CCK8. As can be seen from FIG. 14, for the drug-loaded nanospherical carrier, with the increase of the concentration of the doxorubicin hydrochloride anticancer drug, the cell survival rate decreases gradually, and the cytotoxicity increases. Under the same concentration of the doxorubicin hydrochloride, the carrier coupled with a targeted group has a stronger lethality, indicating that the nanospherical carrier can be used for targeted delivery and release of anticancer drugs to kill cancer cells. In addition, the inventors have found that the oxidized cellulose according to embodiments of the present disclosure has a similar effect on various cancer cells such as hela and MCF-7.

4. Cellular Uptake Experiments

Caco-2 cells were inoculated in a confocal dish, and DO50/DOX was added after the cells were attached to walls of the dish. After a period of time, the cells were washed several times with a phosphate buffer saline (PBS) buffer warmly bathed at 37° C. The cells were fixed with 4% formaldehyde for about 30 min and washed twice with the PBS. Then, a PBS solution containing 0.1% Triton X-100 was added. After 3 to 5 min of standstill at room temperature, excess protein components in cytoplasm were removed. The fixed cells were washed with the PBS, incubated with a PBS solution containing 1% bovine serum albumin (BSA) for 30 min, and then washed again with the PBS for two times. Each sample was added with 3 U of 4′,6-diamidino-2-phenylindole (DAPI)-labeled cell nucleus to react for 20 min, and then washed twice with the PBS. Finally, the sample was observed with a confocal laser scanning microscope (CLSM). The DO50/DOX was excited by 488 nm and 340 nm lasers separately, and confocal fluorescence images at 500-545 nm and 350-395 nm were obtained.

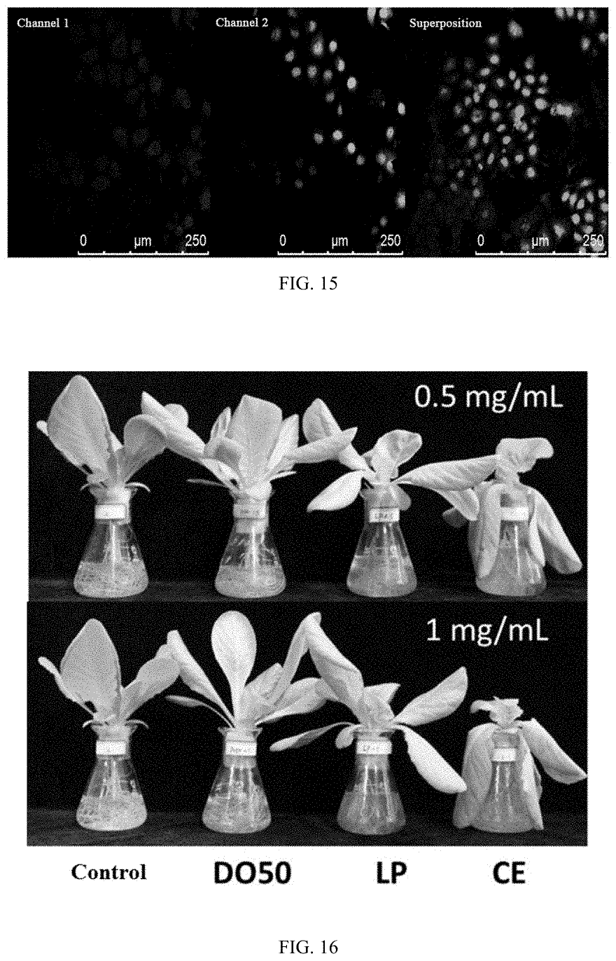

Results are shown in FIG. 15, where channel 1 is a confocal fluorescence image of the DO50/DOX at 488 nm; channel 2 is a confocal fluorescence image of the DAPI at 340 nm; the superposition channel is a superimposed image of channel 1 and channel 2. Experiments show that cellulose nanoparticles are able to adsorb the anticancer drug doxorubicin into cells.

### Example 12

In this example, the oxidized cellulose obtained in Example 1 was studied for phytotoxicity as follows.

For each of the nanospheres (DO50) obtained in Example 1, the laundry powder (LP) and the cleanser essence (CE), 100 mL of sample solutions were prepared separately at a concentration of 0.5 and at 1 mg/mL in triangular flasks. Lettuces with good growth and uniform size were selected and cultured in the flask within an illumination incubator for 48 h under simulated sunlight environment. The growth of the lettuces was photographed with a digital camera, and a photosynthetic rate of leaves of the lettuces was measured at 48 h. For each test group, three parallel experiments were set up, and the control group was only added with water.

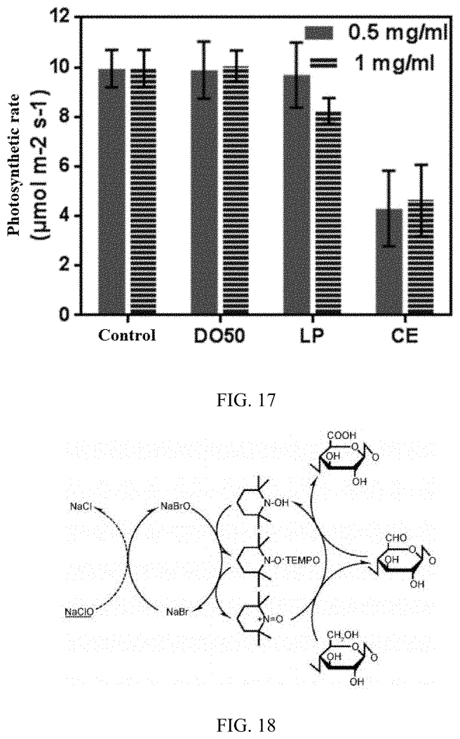

The phytotoxicity is generally reflected by the photosynthetic rate of plant leaves. Experimental results are shown in FIG. 16. As can be seen, the lettuces in the laundry powder and cleanser essence groups are dehydrated and atrophied after 48 h, indicating that the laundry powder and the cleanser essence each are highly toxic to the lettuce. Specifically, it can be further illustrated by FIG. 17 that the photosynthetic rate in each of the laundry powder and cleanser essence groups is significantly lower than that of the control and DO50 groups; while the DO50 does not show obvious toxic damage to the lettuce, indicating that the DO50 is a green non-toxic material.

Reference throughout this specification to “an embodiment,” “some embodiments,” “an example,” “a specific example,” or “some examples,” means that a particular feature, structure, material, or characteristic described in connection with the embodiment or example is included in at least one embodiment or example of the present disclosure. Thus, the appearances of the phrases such as “in some embodiments,” “in one embodiment”, “in an embodiment”, “in another example,” “in an example,” “in a specific example,” or “in some examples,” in various places throughout this specification are not necessarily referring to the same embodiment or example of the present disclosure. Furthermore, the particular features, structures, materials, or characteristics may be combined in any suitable manner in one or more embodiments or examples. In addition, in the absence of contradiction, those skilled in the art can combine the different embodiments or examples described in this specification, or combine the features of different embodiments or examples.

Although explanatory embodiments have been shown and described above, it would be appreciated by those skilled in the art that the above embodiments cannot be construed to limit the present disclosure, and changes, alternatives, variants and modifications can be made in the embodiments without departing from spirit, principles and scope of the present disclosure.

1. A method for preparing oxidized cellulose, comprising:
extracting cellulose from corncob;
preparing the cellulose into a cellulose-contained solution; and
performing 2,2,6,6-tetramethylpiperidinyl-N-oxide (TEMPO) oxidation on the cellulose-contained solution, so as to obtain the oxidized cellulose,
wherein the TEMPO oxidation on the cellulose-contained solution comprises:
mixing TEMPO and NaBr with the cellulose-contained solution, and adjusting pH to 10 with a first NaOH solution, so as to obtain a mixture;
adding a NaClO solution and a second NaOH solution sequentially and alternately to the mixture obtained in step (1) so as to maintain pH of 10; and
adding ethanol to a reaction mixture obtained in step (2) to terminate the reaction, and adding NaBH after 3 to 5 min, so as to obtain the oxidized cellulose,
wherein the TEMPO oxidation is performed at a temperature ranging from 15 to 25° C.

2. The method according to claim 1, wherein based on 1 g of the cellulose, 5 to 12.5 mL, preferably 6.25 mL of the second NaOH solution is consumed in the TEMPO oxidation;
optionally, based on 1 g of the cellulose, 5 to 16 mL of the NaClO solution is consumed in the TEMPO oxidation.

3. The method according to claim 1, wherein based on 1 g of the cellulose, the TEMPO is used in an amount ranging from 0.005 to 0.010 g, preferably 0.008 g.

4. The method according to claim 1, wherein based on 1 g of the cellulose, the NaBr is used in an amount ranging from 0.2 to 0.5 g, preferably 0.4 g.

5. The method according to claim 1, further comprising a purification treatment, comprising:
adjusting an oxidized cellulose-contained reaction mixture obtained in step (3) with a hydrochloric acid solution to pH of 3, and performing a first mixing treatment, so as to obtain a first purified solution;
adjusting the first purified solution with a third NaOH solution to pH of 7, and performing a second mixing treatment, so as to obtain a second purified solution;
adding ethanol to the second purified solution, followed by collecting, washing with ethanol and acetone in sequence, and drying precipitates, so as to obtain the oxidized cellulose,
optionally, extracting cellulose from corncob comprising:
subjecting the corncob to steam explosion and washing corncob residue obtained thereby; and
adding a NaOH solution to the corncob residue to obtain a mixture, followed by heating the mixture, separating solids from the mixture, collecting and washing the solids, so as to obtain the cellulose.

6. An oxidized cellulose, prepared by a method according to claim 1.

7. The oxidized cellulose according to claim 6, wherein the oxidized cellulose is spherical with a particle size ranging from 20 to 30 nm.

8. Use of an oxidized cellulose prepared by a method according to claim 1 in preparation of a detergent, a probe or a drug carrier.

9. (canceled)

10. Use of an oxidized cellulose according to claim 8, wherein the detergent is used at 1 to 10 mg/1 mL water.
